(19) United States
(12) Patent Application Publication
(10) Pub. No.: US 2019/0321367 A1
(43) Pub. Date: Oct. 24, 2019

(54) PALIPERIDONE FOR TREATMENT OF DRUG REFRACTORY IRRITABILITY AND AUTISM SPECTRUM DISORDER

(71) Applicant: Children's Hospital Medical Center, Cincinnati, OH (US)

(72) Inventors: Craig Andrew Erickson, Wyoming, OH (US); Ernest Vijay Pedapati, Cincinnati, OH (US); Logan Wink, Cincinnati, OH (US)

(21) Appl. No.: 16/348,201

(22) PCT Filed: Nov. 21, 2017
(86) PCT No.: PCT/US17/62696

§ 371 Date: May 8, 2019

Related U.S. Application Data
(60) Provisional application No. 62/425,665, filed on Nov. 23, 2016.

(51) Int. Cl. A61K 31/519; A61K 9/16; A61K 9/50; A61K 9/48; A61P 25/18

(52) U.S. Cl. CPC A61K 31/519; A61K 9/16; A61P 25/18; A61K 9/4808; A61K 9/50

(57) ABSTRACT

Disclosed are methods of treating a symptom selected from irritability, self-injurious behavior, intermittent explosive disorder, disruptive behavior disorder, and/or aggression in an individual diagnosed with a Neurodevelopmental Disorder (NDD) such as autism spectrum disorder (ASD). The methods comprise the step of administering to an individual in need thereof a formulation comprising paliperidone.

### CROSS REFERENCE TO RELATED APPLICATIONS

This application claims the benefit of U.S. Application Ser. No. 62/425,665 filed Nov. 23, 2016, which is incorporated herein by reference it its entirety for all purposes.

### BACKGROUND

Neurodevelopmental disorders (NDDs) include Autism and autism spectrum disorder (ASD), intellectual disability (ID), and global developmental delay (GDD)). NDDs comprise of a group of disorders in which the central nervous system disturbance occurs during development which can manifest as impairments in language, non-verbal communication, cognitive abilities, motor function, sensory abnormalities as well as neuropsychiatric diseases. Such patients having an NDD display many symptoms for which effective treatment is lacking. For example, irritability which includes aggression, severe tantrums, and self-injury is a concerning and highly prevalent symptom in autism spectrum disorders, which affects up to 30 to 50% of patients. These prevalences are similar across NDDs at large with an negative correlation with intellectual ability such that estimates of irritability are highest in individuals with similar prevalences across NDD at large (K. McClintock, S. Hall, C. OiiverRisk markers associated with challenging behaviors in people with intellectual disabilities: A meta-analytic study Journal of Intellectual Disability Research, 47 (2003), pp. 405-416; Johnny L. Matson, Christopher Cooper, Carrie J. Malone, Sheree L. Moskow, The relationship of self-injurious behavior and other maladaptive behaviors among individuals with severe and profound intellectual disability, In Research in Developmental Disabilities, Volume 29, Issue 2, 2008, Pages 141-148, ISSN 0891-4222, https://doi.org/10.1016/j.ridd.2007.02.001. (http://www.sciencedirect.com/science/article/pii/S0891422207000170) Keywords: Self-injurious behavior; Intellectual disability; Aberrant Behavior Checklist; Siegel M, Milligan B, Chemelski B, Payne D, Ellsworth B, Harmon J, Teer O, Smith K A. Specialized inpatient psychiatry for serious behavioral disturbance in autism and intellectual disability. J Autism Dev Disord. 2014 Dec;44(12):3026-32. doi: 10.1007/s10803-014-2157-z. PubMed PMID: 24925543.). Presently, second generation antipsychotic agents are very effective, with two agents, risperidone, and aripiprazole, demonstrating a high level of efficacy in placebo controlled trials. These drugs have received FDA approval for the indication of treating irritability with autism spectrum disorders. Despite this, a certain percentage (estimated to be about 30%) of patients do not respond to these drugs (Adler B A, Wink L K, Early M, Shaffer R, Minshawi N, McDougle C J, Erickson C A. Drug-refractory aggression, self-injurious behavior, and severe tantrums in autism spectrum disorders: a chart review study. Autism. 2015 Jan;19(1):102-6. doi: 10.1177/1362361314524641. Epub 2014 Feb 26. PubMed PMID: 24571823.). Drug refractory irritability and autism spectrum disorders can have a high morbidity rate including frequent hospitalizations, medical admissions for self-injury, and danger to caretakers. The instant disclosure seeks to address one or more of the aforementioned needs in the art.

### BRIEF SUMMARY

Disclosed are methods of treating a symptom selected from irritability, self-injurious behavior, intermittent explosive disorder, disruptive behavior disorder, and/or aggression in an individual diagnosed with an autism spectrum disorder (ASD), and other neurodevelopmental disorders (NDDs) which include developmental disorders (DD), intellectual disability (ID), and global developmental delay (GDD).

Where ASD is referred to, it will be understood that the disclosure applies across the spectrum of NDDs.

### DETAILED DESCRIPTION

Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms and expressions used herein have the ordinary meaning as is accorded to such terms and expressions with respect to their corresponding respective areas of inquiry and study except where specific meanings have otherwise been set forth herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms refer to children.

“Therapeutically effective amount” relates to the amount or dose of an active compound or composition described herein that will lead to one or more therapeutic effect, in particular desired beneficial effects. A therapeutically effective amount of a substance can vary according to factors such as the disease state, age, sex, and weight of the subject, and the ability of the substance to elicit a desired response in the subject. Dosage regime may be adjusted to provide the optimum therapeutic response. For example, several divided doses may be administered daily or the dose may be proportionally reduced as indicated by the exigencies of the therapeutic situation.

The phrase “pharmaceutically acceptable,” as used in connection with compositions of the disclosure, refers to molecular entities and other ingredients of such compositions that are physiologically tolerable and do not typically produce untoward reactions when administered to a subject (e.g., human) In certain embodiments, as used herein, the term “pharmaceutically acceptable” means approved by a regulatory agency of a Federal or a state government or listed in the U.S. Pharmacopeia or other generally recognized pharmacopeia for use in mammals (e.g., humans).

The term “carrier” applied to pharmaceutical compositions of the disclosure refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like. Suitable pharmaceutical carriers are described in “Remington's Pharmaceutical Sciences” by E. W. Martin, 18th Edition.

The term “compound,” as used herein, is also intended to include any salts, solvates, or hydrates thereof.

The terms “pharmaceutically acceptable salts” or “a pharmaceutically acceptable salt thereof” refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as The Merck Index. Any suitable constituent can be selected to make a salt of an active drug discussed herein, provided that it is non-toxic and does not substantially interfere with the desired activity. In addition to salts, pharmaceutically acceptable precursors and derivatives of the compounds can be employed. A salt of a compound of this disclosure may be formed between an acid and a basic group of the compound, such as an amino functional group, or a base and an acidic group of the compound, such as a carboxyl functional group. According to another embodiment, the compound is a pharmaceutically acceptable acid addition salt. Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

Neurodevelopmental disorders (NDD) comprise of a group of disorders in which the central nervous system disturbance occurs during development which can manifest as impairments in language, non-verbal communication, cognitive abilities, motor function, sensory abnormalities as well as neuropsychiatric diseases. Such patients having an NDD display many symptoms for which effective treatment is lacking. For example, irritability, aggression, and self-injury is a concerning and highly prevalent symptom in autism spectrum disorders, which affects up to 30 to 50% of patients.

A specific NDD, include Autism Spectrum Disorder (ASD) which the core symptoms include dysfunction in social interaction, communication, and repetitive behavior. A severe and common (>30%) co-morbidity is irritability, which can be characterized by severe tantrums, self-injury, physical aggression, and emotional dysregulation. Irritability in previous clinical trials have been defined as manifesting clinically significant problems consisting of aggression, tantrums, and/or self-injury as defined with a score of 18 or higher on the irritability subscale of the Aberrant Behavior Checklist (Aman, Michael G., Will H. Burrow, and Patricia L. Wolford. “The Aberrant Behavior Checklist-Community: factor validity and effect of subject variables for adults in group homes.” American Journal on Mental Retardation (1995)) rated by a parent or caretaker and confirmed by a clinician. In addition, subjects can be clinically rated by the Clinical Global Impression (CGI) severity scale with a score of moderate or higher from the clinician. Currently, the atypical antipsychotics risperidone and aripiprazole have been approved by the US Food and Drug Administration (FDA) for the treatment of irritability in youth with ASD. (Shea 2004, Hellings 2006, Marcus 2009, Owen 2009). Both risperidone and aripiprazole are extensively metabolized by CYP2D6 and CYP3A4. In the case of risperidone, this causes a release of a highly efficacious active metabolite, 9-hydroxyrisperidone (paliperidone).

Polymorphisms of CYP2D6 (accession number M33388) and CYP3A4 (accession number P08684) are involved in the oxidative metabolism of risperidone and aripiprazole (Zhou, 2009). The CYP designation is such that the first numeral defines the family, the second character defines the subfamily, and the final numeral defines the gene product. CYP2D6, in particular, is estimated to affect the pharmacokinetics of 50% of drugs used in clinical practice (Zhou, 2009). Polymorphisms in these enzymes can influence enzyme activity ranging from complete loss of catalytic activity (poor metabolizer; PM) to increased enzyme activity associated with gene duplication (ultra-rapid metabolizer, UM). The distribution frequency of the different CYP2D6 deficient and increased activity alleles varies among different ethnic population with up to 7% to 10% of Caucasians are categorized as PMs (Sistonen et al., 2007).

The impact of major cytochrome P450 (CYP) polymorphisms as a cause of inter-individual variability of response to psychiatric medications in individuals with autism or other developmental disabilities have not been systematically studied (Buitelaar, 2003). In general, CYP isoenzymes may modulate clinical response to a medication by either catalyzing the inactivation of an active drug or increasing activity by catalyzing a prodrug into an active drug. The evidence for the use of pharmacogenetics to enhance clinical practice is still emerging (Dunnenberger et al., 2015).

As individuals may have different forms or amounts of CYPs, a personalized medicine approach using CYP testing has been successfully used in pain management where the analgesic effects and ADR of several commonly used opiates can be primarily explained by liver metabolism (Crews et al., 2014; Vuilleumier, Stamer, & Landau, 2012). In other conditions, the evidence for the use of pharmacogenetics is less compelling (Fleeman et al., 2011; Roberts, Mulder, Joyce, Luty, & Kennedy, 2004; Shi et al., 2015).

Three specific cytochrome P450 (CYPs) 2D6, 3A4, and 1A2 (accession number P05177) (see, e.g., Nelson D R, Koymans L, Kamataki T, Stegeman J J, Feyereisen R, Waxman D J, Waterman M R, Gotoh O, Coon M J, Estabrook R W, Gunsalus I C, Nebert D W. P450 superfamily: update on new sequences, gene mapping, accession numbers and nomenclature. Pharmacogenetics. 1996 Feb;6(1):1-42. Review. PubMed PMID: 8845856.) have a special relevance in psychiatry as they affect the clearance of most antipsychotics through oxidative metabolism which involves the phase I conversion of a parent drug. There at least 17 mammalian CYP gene families and more than 30 human gene products have been identified (Nelson 1996). CYP isoforms may be predictive of adverse drug reactions (ADRs), such as tardive dyskinesia (Patsopoulos, Ntzani, Zintzaras, & Ioannidis, 2005; Zhou, 2009). In a study of 325 adult patients taking risperidone, for example, the odds ratio for individuals with poor CYP2D6 metabolism (n=27) when compared to non-poor metabolizers were highly associated with reporting moderate to severe ADRs (OR=3.1) and were more likely to discontinue risperidone due to an ADR (OR=3.4) (J. de Leon et al., 2005).

Of particular interest, a certain percentage of refractory NDD patients are CYP2D6/3A4 slow metabolizers. As such, this subset of patients would benefit from direct administration of the active metabolite that bypasses the CYP2D6 oxidative pathway, namely paliperidone, which, in clinical trials has similar efficacy to risperidone. In particular, CYP2D6 isoforms *1, *2, *33, and *35 have normal enzymatic activity, *10 and *41 have reduced activity, and *3, *4, *5 have no activity. If there is a gene duplication of a functional gene copy, this can result increased activity. These “functional” phenotypes include poor metabolizers (PM), intermediate metabolizers (IM), extensive metabolizers (EM), and ultrarapid metabolizers (UM) based on the expression of these alleles. Once the isoforms are identified, they can be converted into an activity score which reflects the relative activity of the CYP2D6 activity in a particular patient (Gaedigk, A., Simon, S., Pearce, R., Bradford, L., Kennedy, M. and Leeder, J. (2008), The CYP2D6 Activity Score: Translating Genotype Information into a Qualitative Measure of Phenotype. Clinical Pharmacology & Therapeutics, 83: 234-242. doi:10.1038/sj.clpt.6100406). This problem was recently summarized by Vanwong et al. 2017 looking at the impact of CYP2D6 polymorphism on the steady state plasma levels of risperidone and paliperidone in children with ASD. In this study, they identified that levels of the parent compound were strongly influenced by the CYP2D6 polymorphisms. There was also an increased risperidone/paliperidone ratio, but the paliperidone levels were not influenced (which replicated previous studies in adults). Higher plasma levels of risperidone are associated with increased risk for adverse effects such as extrapyramidal symptoms. Thus, Applicant has found that in a certain subset of the ASD population having reduced Phase I metabolism of risperidone, such population may have a more favorable response to paliperidone.

Disclosed herein are methods of treating a symptom selected from irritability, self-injurious behavior, intermittent explosive disorder, disruptive behavior disorder, aggression, or combinations thereof in an individual diagnosed with an autism spectrum disorder (ASD). The methods may comprise the step of administering to an individual a formulation comprising paliperidone having the following structure

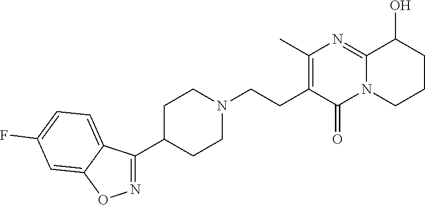 [SMILES: Cc1nc2n(c(=O)c1CCN1CCC(c3noc4cc(F)ccc34)CC1)CCCC2O]

or a pharmaceutically acceptable salt thereof.

In one aspect, the paliperidone or pharmaceutically acceptable salt thereof may be in a form selected from a micropellet, a powder and a granule.

In one aspect, the individual may be administered a dose of about 1.5 to about 18 mg/day, or about 3 to about 15 mg/day, or about 5 to about 12 mg/day.

In one aspect, the paliperidone or pharmaceutically acceptable salt thereof may be administered at an interval selected from twice a day, three times a day, or four times a day.

In one aspect, the paliperidone or pharmaceutically acceptable salt thereof may be in the form of a sprinkle dosage form, and may comprise an openable capsule enclosing a plurality of micropellets. In one aspect, the micropellets may be taste-masked.

In one aspect, method of diagnosing and treating a symptom selected from irritability which may include self-injurious behavior, aggressive behavior, tantrums marked with excessive duration or severity is disclosed. Irritability may occur as an independent mental state or occur in the course of other psychiatric conditions (including International Classification Code Numbers) including intermittent explosive disorder (F63.9), bipolar affective disorder (F31), disruptive behavior disorder (F98.9), major depressive disorder (F33.2), aggression (F45.6), catatonic disorder (F06.1), and psychotic disorders such as schizophrenia (F20) in an individual diagnosed with a Neurodevelopmental Disorder (NDD) such as autism spectrum disorder (ASD). Irritability in previous clinical trials have been defined as manifesting clinically significant problems consisting of aggression, tantrums, and/or self-injury as defined with a score of 18 or higher on the irritability subscale of the Aberrant Behavior Checklist (Aman 1995) rated by a parent or caretaker and confirmed by a clinician. In addition, subjects can be clinically rated by the Clinical Global Impression (CGI) severity scale with a score of moderate or higher from the clinician.

The method may comprise the steps of detecting whether a mutation exists in the individual that indicates reduced CYP2D6 and/or CYP3A4 activity; diagnosing the patient as a candidate for paliperidone treatment when the presence of the mutation is detected; and administering an effective amount of paliperidone or pharmaceutically acceptable salt thereof to the diagnosed patient.

In one aspect, a method of diagnosing and treating a symptom selected from irritability which may include self-injurious behavior, aggressive behavior, tantrums marked with excessive duration or severity. Irritability may occur as an independent mental state or occur in the course of other psychiatric conditions (including International Classification Code Numbers) including intermittent explosive disorder (F63.9), bipolar affective disorder (F31), disruptive behavior disorder (F98.9), major depressive disorder (F33.2), aggression (F45.6), catatonic disorder (F06.1), and psychotic disorders such as schizophrenia (F20) in an individual diagnosed with a Neurodevelopmental Disorder (NDD) such as autism spectrum disorder (ASD) is disclosed. The method may comprise the steps of obtaining a biological sample from an individual in need of such treatment; detecting the activity of CYP2D6 and CYP3A4 in said individual; diagnosing the individual as a candidate for paliperidone treatment when the activity of CYP2D6 and/or CYP3A4 is reduced or expected to be reduced based on the presence of a mutation in CYP2D6 and/or CYP3A4 that is expected to disrupt function of CYP2D6 and/or CYP3A4; and administering an effective amount of paliperidone or a pharmaceutically acceptable salt thereof, to the diagnosed individual, the dose being determined via a dose titration consistent with typical clinical care as understood by one of ordinary skill in the art.

CYP isoforms can be measured by a variety of commercially available kits. Mutations may include any isoform of *9, *10, *17, *29, *41, *49, *50, *54, *55, *59, *69, *72 which indicates reduced activity of CYP2D6 or *3, *4, *5, *6, *7, *8, *11, *12, *13, *14, *14A, *14B, *15, *16, *18, *19, *20, *21, *31, *36, *38, *40, *42, *44, *47, *51, *56, *56A, *56B, *57, *62 which indicate no activity. In one aspect, the mutation in CYP2D6 or CYP3A4 may be one in which there is a reduced capacity to metabolize risperidone and/or aripiprazole in the subject. Where the patient is identified as being a candidate for paliperidone treatment, the paliperidone may be in the form of a sprinkle dosage form (powder) as described herein. In one aspect, the paliperidone is administered at an interval selected from once a day, twice a day, three times a day, or four times a day. In one aspect, the sprinkle dosage form comprises a capsule enclosing a plurality of micropellets, wherein the capsule has a means for opening the capsule. In one aspect, the micropellets are taste-masked.

The biological sample obtained for determination of a mutation may include, for example, saliva, blood, or skin, or any other biological source as would be readily appreciated by one of ordinary skill in the art.

In one aspect, a kit is disclosed. The kit may comprise paliperidone or pharmaceutically acceptable salt thereof and a pharmaceutically acceptable carrier; and a means for delivery of the composition to a human. In one aspect, the paliperidone or pharmaceutically acceptable salt thereof in the kit may be in the form of a sprinkle composition.

In one aspect, an article of manufacture is disclosed. In this aspect, the article of manufacture may comprise: a container comprising a label; and a composition comprising paliperidone or pharmaceutically acceptable salt thereof and a pharmaceutically acceptable excipient, wherein the label indicates that the composition is to be administered to an individual affected by an NDD (including ASD) is diagnosed with irritability which may include self-injurious behavior, aggressive behavior, tantrums marked with excessive duration or severity. Irritability may occur as an independent mental state or occur in the course of other psychiatric conditions (including International Classification Code Numbers) including intermittent explosive disorder (F63.9), bipolar affective disorder (F31), disruptive behavior disorder (F98.9), major depressive disorder (F33.2), aggression (F45.6), catatonic disorder (F06.1), and psychotic disorders such as schizophrenia (F20) in an individual diagnosed with a Neurodevelopmental Disorder (NDD) such as autism spectrum disorder (ASD).

Sprinkle Dosage Forms

Sprinkle dosage forms include particulate or pelletized forms of paliperidone, optionally having functional or non-functional coatings, with which a patient or a caregiver can sprinkle the particulate/pelletized dose into drink or onto food. A sprinkle dosage form may comprise particles of about 10 to about 100 micrometers in their major dimension. Sprinkle dosage forms may be in the form of optionally coated granules or as microcapsules. Sprinkle dosage forms may be immediate or controlled-release formulations such as sustained-release formulations. See U.S. Pat. No. 5,084,278, which discloses microcapsule formulations, which may be administered as sprinkle dosage forms.

The dosage forms of paliperidone can comprise uncoated particulates, granules, and/or powders of paliperidone, particulates, granules, and/or pellets of paliperidone coated with a highly soluble immediate release coating. Such coatings include, for example, Opadry® type coating, as are known to those skilled in the art (see, e.g., U.S. Pat. No. 5,098,715). Inert cores may also be used, and can comprise any appropriate type of core material useful in pharmaceutical applications, which can be water insoluble, such as cellulose spheres or silicon dioxide, or can be water soluble such as starch and/or sugar. A core can comprise beads, ion-exchange resin beads, spheroids, spheres, seeds, pellets, granules, or other particulate form. A core can comprise a material such as sugar, starch, sugar and starch, sucrose crystals, extruded and dried spheres comprising excipients such as microcrystalline cellulose and lactose, or an acidic or alkaline buffer crystal such as calcium carbonate, sodium bi-carbonate, fumaric acid, tartaric acid which can alter the microenvironment of the drug to facilitate release of the drug. In certain embodiments, a core comprises sugar (Sugar Sphere NF). A core can have any appropriate dimension suitable for oral delivery. For example, a core can have a diameter ranging from about 15 mesh to about 50 mesh, from about 20 mesh to about 25 mesh, or from about 30 mesh to about 35 mesh. In certain embodiments, the diameter of a core can range from about 0.25 mm to about 3 mm, and in certain embodiments, from about 0.5 mm to about 1 mm

Examples of coating materials for effecting controlled or modified release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropyl methyl cellulose phthalate, methylcellulose, ethyl cellulose, cellulose acetate, cellulose acetate phthalate, cellulose acetate trimellitate, and carboxymethylcellulose sodium; acrylic acid and methacrylic acid copolymers, methyl methacrylate copolymers, ethoxy-ethyl methacrylates, cyanoethyl methacrylate, poly(acrylic acid), poly(methacrylic acid), meth-acrylic acid alkylamide copolymer, poly(methyl methacrylate), polymethacrylate, poly(methyl methacrylate) copolymers, polyacrylamide, aminoalkyl methacrylate copolymer, poly(methacrylic acid anhydride), glycidyl methacrylate co-polymers, ammonio methacrylate copolymers, and methacrylic resins commercially available under the tradename Eudragt™ including Eudragit™ L, Eudragit™ S, Eudragit™ E, Eudragit™ RL, and Eudragit™RS; vinyl polymers and copolymers such as polyvinylpyrrolidone, vinyl acetate, vinylacetate phthalate, vinylacetate crotonic acid co-polymer, and ethylene-vinyl acetate copolymer; enzymatically degradable polymers such as azo polymers, pectin, chitosan, amylase, and guar gum; and shellac. Combinations of any of the foregoing polymers may also be used to form a controlled-release coating. In certain embodiments, more than one controlled release coating can be used to modify the release properties of paliperidone from a particle.

In certain embodiments, a controlled-release coating can provide pH-dependent release of paliperidone in the gastrointestinal tract. A pH-dependent coating may be designed to release paliperidone from a particle in a desired area or areas of the gastrointestinal tract such as the small intestine and/or colon depending on the pH of the gastrointestinal fluid in the desired area of the gastrointestinal tract. Examples of pH-dependent coating materials useful in certain embodiments include shellac, cellulose acetate phthalate, polyvinyl acetate phthalate, hydroxypropyl methylcellulose phthalate, and methacrylic acid ester copolymers, zein, and the like. In certain embodiments, a pH-dependent coating can be a copolymer synthesized from diethylaminoethyl methacrylate and other neutral methacrylic esters, also known as methacrylic acid copolymers or polymer methacrylates, commercially available as Eudragit™ (Rohm Pharma). There are several different types of Eudragit™ polymers useful for imparting pH-dependent release properties. For example, Eudragit™ E is a methacrylic copolymer that swells and dissolves in acidic media. Eudragit™ L is a methacrylic acid copolymer that does not swell at about pH<5.7 and is soluble at about pH>6. Eudragit™ S does not swell at about pH<6.5 and is soluble at about pH>7. Eudragit™ S and Eudragit™ L can be used as single components in a controlled-release coating or in a combination in any ratio to achieve desired release properties. By using a combination of copolymers, a controlled release coating can exhibit a solubility at a pH between the pHs at which, for example, Eudragit™ S and Eudragit™ L are separately soluble.

In certain embodiments, a controlled-release coating can provide pH-independent release of paliperidone in the gastroinetestinal tract. Ammonioalkyl methacrylate copolymers such as Eudragit™ RS and Eudragit™ RL are examples of useful pH-independent polymers. Eudragit™ RL and Eudragit™ RS are acrylic resins comprising copolymers of acrylic and methacrylic acid esters with a low content of quaternary ammonium groups. The ammonium groups are present as salts and impart permeability to the lacquer films. Eudragit™ RL and Eudragit™ RS swell in water and digestive juices, in a pH-independent manner In the swollen state coatings formed there-from are permeable to water and release or dissolve active compounds. Eudragit™ RL and Eudragit™ RS can be used alone in a pH-independent coating, combined together, combined with other ammonioalkyl methacrylate copolymers, or other methacrylic acid copolymers to achieve a desired release property.

In certain embodiments, a controlled-release coating can comprise a pharmaceutically acceptable acrylic polymer including, for example, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymers, polyacrylamide, aminoalkyl methacrylate copolymer, poly(methacrylic acid anhydride), glycidyl methacrylate co-polymers, ammonioalkyl methacrylate copolymers, and combinations of any of the foregoing.

A coating comprising paliperidone and/or other coating used to form immediate- or controlled-release particles provided by the present disclosure such as a sealant coating, a barrier coating, a pH-dependent release coating, and a pH-independent release coating can also comprise one or more pharmaceutically acceptable excipients such as, for example, surfactants, lubricants, diluents, plasticizers, anti-adherents, glidants, buffers, disintegrants, fillers, wetting agents, emulsifying agents, pH buffering agents, pH-modifying agents, stabilizing agents, chelating agents, binders, thickening agents, coloring agents, and combinations of any of the foregoing.

Examples of surfactants useful in pharmaceutically acceptable coatings provided by the present disclosure include pharmaceutically acceptable anionic surfactants, cationic surfactants, amphoteric (amphiphatic/ampophilic) surfactants, nonionic surfactants, polyethyleneglycol esters or ethers, and combinations of any of the foregoing. Examples of useful pharmaceutically acceptable anionic surfactants include monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates such as sodium lauryl sulfate, ethoxylated alkyl sulfates, ester linked sulfonates such as docusate sodium and dioctyl sodium succinate, alpha olefin sulfonates, or phosphated ethoxylated alcohols. Examples of pharmaceutically acceptable cationic surfactants useful in coatings provided by the present disclosure include monoalkyl quaternary ammonium slats, dialkyl quaternary ammonium compounds, amidoamines, and aminimides. Examples of useful pharmaceutically acceptable amphoteric surfactants include N-substituted alkyl amides, N-alkyl betaines, sulfobetaines, and N-alkyl-6-aminopropionates. Examples of pharmaceutically acceptable polyethyleneglycol esters or ethers useful in coatings provided by the present disclosure include polyethoxylated castor oil, poly-ethoxylated hydrogenated castor oil, or hydrogenated castor oil.

Lubricants and anti-static agents may be included in a pharmaceutically acceptable coating to aid in processing. Examples of lubricants useful in coatings provided by the present disclosure include calcium stearate, glycerol behenate, glyceryl monostearate, magnesium stearate, mineral oil, polyethylene glycol, sodium stearyl fumarate, sodium lauryl sulfate, stearic acid, talc, vegetable oil, zinc stearate, and combinations of any of the foregoing. In certain embodiments, the lubricant is glyceryl monostearate. In certain embodiments, coatings may comprise an amount of lubricant ranging from about 1 wt % to about 15 wt % based on the total solids weight of the coating.

Plasticizers may be included in pharmaceutically acceptable coatings to improve the physical properties of the cured coating. For example, plasticizers may increase the flexibility or elasticity of a coating comprising a film-forming material having a relatively high glass transition temperature such as ethyl cellulose. Examples of plasticizers useful in coatings provided by the present disclosure include adipates, azelate, benzoates, citrates, isoebucates, phthalates, sebacates, stearates, glycols, polyethylene glycol, propylene glycol, triacetin, dimethyl phthalate, diethyl phthalate, dibutyl phthalate, dibutyl sebacate, triethyl citrate, tributyl citrate, triethyl acetyl citrate, acetyltributylcitrate, glyceryl triacetate, castor oil, acetylated monoglycerides, and combinations of any of the foregoing.

Glidants may be included in pharmaceutically acceptable coatings to reduce sticking effects during processing, film formation, and/or drying. Examples of glidants useful in coatings provided by the present disclosure include talc, magnesium stearate, glycerol monostearate, colloidal silicon dioxide, precipitated silicon dioxide, and combinations of any of the foregoing.

Examples of pH-buffering agents useful in coatings provided by the present disclosure include citric acid, sodium citrate, fumaric acid, sodium fumarate, and combinations of any of the foregoing.

pH modifying agents can create a microenvironment around paliperidone when exposed to aqueous fluids, thereby enhancing its absorption through the intestinal epithelia. Examples of pharmaceutically acceptable alkaline pH modifying agents include, for example, L-lysine, L-arginine, sodium citrate, and magnesium hydroxide. Examples of pharmaceutically acceptable acidic pH modifying agents include, for example, fumaric acid, citric acid, tartaric acid, malic acid, maleic acid, and succinic acid.

Examples of stabilizers useful in coatings provided by the present disclosure include antioxidants such as 3,5-di-tert-butyl-4-hydroxytoluene (BHA), 3-(or 2)-tert-butyl-4-hydroxyanisole (BHT), ascorbic acids, tocopherols, and the like.

Binders may be included in coating compositions to hold the components of a coating together. Examples of binders useful in coatings provided by the present disclosure include, for example, polyvinylpyrrolidone, hydroxypropyl cellulose, hydroxypropyl methylcellulose, methylcellulose, hydroxyethyl cellulose, sugars, dextran, cornstarch, and combinations of any of the foregoing. In certain embodiments, the binder is polyvinylpyrrolidone such as Plasdone® K29/32 Plasdone (ISP Technologies, Wayne, N.J.).

Anti-foaming agents may also be included in pharmaceutically acceptable coatings. Examples of anti-foaming agents useful in coatings provided by the present disclosure include silicone and simethicone.

Examples of pigments useful in coatings provided by the present disclosure include titanium dioxide, food color lakes, and iron oxides.

Controlled release coatings provided by the present disclosure may also comprise erosion-promoting agents such as starch and gums, materials useful for making microporous lamina in the use environment such as polycarbonates characterized by linear polyesters of carbonic acid in which carbonate groups reoccur in the polymer chain, and/or semi-permeable polymers such as hydroxypropylmethyl cellulose, lactose, and metal stearates.

Release-modifying materials, which may be incorporated into controlled release coatings, and which function as pore-formers may be organic or inorganic, include materials that can be dissolved, extracted, or leached from the coating in the environment of use, e.g., the gastrointestinal tract or in a certain region or regions of the gastrointestinal tract, such as hydrophilic materials, for example, hydroxypropylmethyl cellulose.

The combination of all solid components of the polymeric material, including co-polymers, fillers, plasticizers, and optional excipients and processing aids, can provide an about 10% to about 450% weight gain to the cores. In certain embodiments, the weight gain is about 30 to about 160%.

Any of the controlled-release materials can be combined with one or more other controlled release materials to provide a controlled-release coating, or can be provided as separate coatings, each coating comprising one or more controlled-release materials applied to a core comprising a coating of paliperidone.

Dosage

As will be apparent to those skilled in the art, dosages outside of these disclosed ranges may be administered in some cases. Further, it is noted that the ordinary skilled clinician or treating physician will know how and when to interrupt, adjust, or terminate therapy in consideration of individual patient response.

In certain embodiment, the dosage of the composition provided herein, based on weight of the active compound, administered to prevent, treat, manage, or ameliorate a disorder, or one or more symptoms thereof in a subject may be about 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is a unit dose of about 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In one aspect, the active ingredient or a pharmaceutically acceptable salt thereof, may be present in an amount of from about 0.5% to about 95%, or from about 1% to about 90%, or from about 2% to about 85%, or from about 3% to about 80%, or from about 4%, about 75%, or from about 5% to about 70%, or from about 6%, about 65%, or from about 7% to about 60%, or from about 8% to about 55%, or from about 9% to about 50%, or from about 10% to about 40%, by weight of the composition.

### EXAMPLES

Methods: Applicant performed a retrospective chart review of the treatment course of three youth with ASD and irritability who were treated with paliperidone based on pharmacogenetics testing after failure of first-line treatments. In each case, the patient was cross titrated onto paliperidone after treatment failure with aripiprazole or risperidone. Clinical severity at baseline and improvement with treatment was measured using the Clinical Global Impression Severity scale (CGI-S) and Clinical Global Impressions Improvement scale (CGI-I).

Results: One female and two males diagnosed with ASD and co-morbid psychiatric diagnoses received paliperidone treatment based on CYP genotype indicating a poor metabolizer of risperidone and aripiprazole. The mean dose of final dose paliperidone based on weight was 0.15 mg/kg. Reported adverse effects including appetite increase, however, no extrapyramidal symptoms was noted. Mean CGI-S at baseline was 4.6. Caregiver reports indicated clinical improvement in target symptoms with a mean follow up CGI-I score of 1.7.

Conclusion: This case series provides clinical examples for the use of pharmacogenetics to individualize treatment in a specialized clinical population with a high risk of severe behaviors. The implications of these results including incorporation of rapid, specific testing of CYP P450 2D6 activity level to target paliperidone is discussed. In addition, additional considerations for paliperidone reformulation to optimize effectiveness in ASD is disclosed.

In the following case series, Applicant identified the impact of targeting paliperidone to treat refractory severe behavior based on pharmacogenetics testing in three youth with autism who failed risperidone or aripiprazole.

Subjects and Clinical Measures

This was a prospectively derived retrospective clinical cohort study of patients conducted within Cincinnati Children's Hospital Medical Center (CCHMC) to evaluate the role of pharmacogenetics testing in the treatment of refractory irritability in youth with ASD. Cases were identified from a database of patients with ASD who were treated for irritability between January 2015 and March 2016. Patients who receive a chart diagnosis of ASD at CCHMC have undergone a rigorous interdisciplinary assessment including clinical psychological testing and diagnosis by a physician. Epidemiological, demographic, medical history, clinical outcome, and adverse effects data was collected for analysis. As part of routine practice, a Clinical Global Impression severity (CGI-S) and improvement (CGI-I) score was rated by the treating child psychiatrist for each visit (Guy, 1976) by Applicant. The CGI-S and CGI-I were designed for clinicians to quantify and track patient progress and treatment response over time. Therapeutic response was identified by CGI-I score of 3. Serious adverse event (SAE) was defined as new on-set extrapyramidal symptoms and hospitalization. The study was approved by the institutional IRB. All data were exported from the medical record in a de-identified format for analysis.

Inclusion Criteria

Incident cases were identified from the database with an outpatient encounter with the following diagnostic codes (ICD-10): irritability, self-injurious behavior, intermittent explosive disorder, disruptive behavior disorder, and aggression. Applicant identified potential subjects with ASD and who were taking an antipsychotic for irritability with available genetic testing.

Genotype Testing

Pharmacogenetics testing was ordered by the treating physician over the course of clinical treatment and scanned into the medical records. GeneSight Psychotropic testing (AssureRx Health, Inc., Mason, Ohio) was used in all cases. DNA was collected using a buccal swab and sent to AssureRx for amplification of relevant genomic regions by polymerase chain reaction. Analysis of CYP1A2, CYP2B6 (A785G), CYP2B6 (G516T), CYP2C19, CYP2C9, CYP3A4 and HTR2A was completed by using a custom xTAG® assay (Luminex Molecular Diagnostics). Per the manufacture, the following genetic variants were able to be detected in the assay: CYP1A2-3860G>A, -2467T>delT, -739T>G, -729C>T, -163C>A, 2116G>A, 2499A>T, 3497G>A, 3533G>A, 5090C>T, 5347C>T; CYP2B6 *1, *4, *6, *9; CYP2C19 *1, *2, *3, *4, *6, *8, *17; CYP2C9 *1, *2, *3, *5, *6; CYP2D6 *1, *2, *2A, *3, *4, *5, *6, *7, *8, *9, *10, *11, *12, *14, *15, *17, *41, gene duplication; CYP3A4 *1, *13, *15A, *22. Based on previous methods Applicant calculated a 2D6 “activity score” which is a method to predict individual phenotypic response to polymorphic cytochrome P450 genotype (Gaedigk et al., 2008). Specifically, an activity score of 0.5 indicates an intermediate metabolizer and 0 indicates a poor metabolizer.

Patient 1

Patient 1 was a 15-year-old male with a history of intermittent explosive disorder, attention deficit disorder, and irritability in the context ASD with moderate intellectual disability. The presenting complaint by the caregiver was extreme temper outbursts which resulted in physical aggression, self-injury, and property destruction. Additional concerns including a poor attention span, not listening when spoken to, and easy distractibility. Many symptoms were reported to be present daily, with extreme agitation approximately once a week. At the initial visit the patient was on extended release dexmethylphenidate 25 mg PO daily, dexmethylphenidate 10 mg every afternoon, extended release guanfacine 3 mg PO daily, risperidone 1 mg PO BID, and sertraline 75 mg PO daily. The referral had indicated that prior to starting risperidone, the patient failed a trial of aripiprazole 5 mg PO BID which was reported to cause worsening agitation. After the initial consultation, he was rated by the psychiatrist with a CGI-S of “5-markedly ill” and the plan was to titrate risperidone to address the symptoms of irritability. Over the course of several months, the patient was titrated to risperidone 2 mg PO TID. During the titration he developed extrapyramidal symptoms which resolved after starting benzotropine 0.5 mg PO BID. The patient continued to be symptomatic, including several expulsions from school for physical aggression. In an effort to address explosive behavior, the patient had a trial of valproic acid which was titrated to a serum trough level of 103.2. Though initially the patient was mildly sedated which seemed to improve behaviors, the caregiver felt the patient's severe behaviors were unchanged. The patient also developed bilateral hand tremors, which resolved following discontinuation of valproic acid. Due to refractory irritability, pharmacogenetics testing was obtained which revealed reduced CYP2D6 activity (*4/*9) consistent with a poor metabolizer. The patient was cross titrated from risperidone to paliperidone 9 mg PO daily. The caregiver noted a marked and dramatic improvement in target symptoms. He had noted that for many years, the patient would repeatedly bang his head on the bathroom wall during his morning care. This behavior was eliminated after starting paliperidone. Overall, the patient was reported to be calmer with less frequent and severe tantrums. The patient was continued on benzotropine, with no emerging movements. Following the initiation of paliperidone, his CGI-S rating was unchanged with a CGI-I rating noted to be “1-very much improved”.

Patient 2, AP

Patient 2 was a 14-year old African American male with a history of intermittent explosive disorder, ADHD, obsessive compulsive disorder, mixed receptive-expressive language disorder in the context of Autism Spectrum Disorder (formally Pervasive Developmental Disorder NOS) with mild intellectual disability (FSIQ=59) referred for medication management for irritability. Since early childhood, the patient struggled with repetitive behavior which interfered with his functioning (i.e. repeatedly erasing the chalkboard and spinning parts of objects). However, over the last several years, the caregivers have reported that the patient will become upset and physically aggressive if these behaviors are interrupted. In the mornings, for example, the patient would use foul language and kick his father during morning cares. Both caregivers demonstrated multiple bruises on their extremities which were attributed to the patient's physical aggression which was at times unprovoked. In addition, he had poor attention and was listless in most environments. Despite accommodations at school including a specialized aide for the patient and a modified curriculum, the patient would frequently be unable to finish his work and would become upset in the classroom. The patient was referred by a psychologist to a child psychiatrist from a specialized, intense outpatient behavioral treatment program for severe behaviors. Upon initial outpatient presentation, the patient was on aripiprazole 30 mg PO daily, methylphenidate ER 60 mg PO daily, benzotropine 0.25 mg PO BID, guanfacine 0.5 mg PO BID, and metformin sustained release 500 mg PO daily. Previously prior to switching to aripiprazole, the patient had a trial of risperidone up to 0.5 mg PO three times daily, but there was no significant improvement in irritability and significant weight gain. The caregivers felt the aripiprazole was moderately beneficial, but had only noticed the difference as they increased the total daily dose over 20 mg. At the higher doses, the patient developed extrapyramidal movements which resolved with benzotropine. However, the patient remained highly irritable and aggressive during behavioral therapy visits. The psychiatrist noted that the patient would frequently and suddenly hit his father even during the office visit. Prior to consideration of switching to another atypical antipsychotic or typical antipsychotic the psychiatrist obtained pharmacogenetics testing. The test results indicated a CYP2D6 *4/*17 genotype suggesting a poor metabolizer phenotype. Over a two-week period, the patient was cross titrated from aripiprazole to paliperidone 9 mg daily. Following the transition to paliperidone, the caregivers reported a steady reduction in physical aggression and irritability. In the room, the psychiatrist noted the patient was more restrained in his frustration and using foul language but did not become physical. There was no change in the patient's attention and hyperactivity. There was no change in the frequency in which he engaged in repetitive behaviors, however, he could be more easily redirect with less severe tantrums. The CGI-S was unchanged at “4-moderately ill” and the psychiatrist rated the improvement with a CGI-I score of “2-much improved”.

Patient 3

Patient 3 was a 9-year-old female with Fragile X syndrome (FXS), a monogenetic disorder associated with ASD and the most common cause of inherited intellectual disability. She additionally has a history of intellectual disability (mild to moderate), ASD, anxiety, and intermittent explosive disorder. She presented for treatment of significant irritable tantrums, physical aggression, and self-harming behaviors. She was maintained on sertraline 150 mg daily and trazodone 100 mg throughout the course of her treatment. The initial CGI-S score was rated by the clinician as “5-markedly ill”. She failed an aripiprazole titration to 7.5 mg whose use was associated with significant AEs including sedation and lethargy. Following the results of pharmacogenetics testing, she was noted to be a poor metabolizer of risperidone and aripiprazole (2D6 activity score: 0) she began a trial of paliperidone 1.5 mg PO daily. After several dose adjustments over two months to address ongoing irritability, she maintained a 6 mg paliperidone daily dose. Paliperidone use was associated with significant improvements in irritable moods, self-injury, and physical aggression. At follow up visit two months following initiation of paliperidone treatment her CGI-I score was scored as “2-much improved.”

In addition to these cases, Applicant have identified through a chart review over 20 cases of patients with abnormal CYP2D6/3A4 activity that poorly tolerated aripiprazole/risperidone and had a favorable clinical outcome when switched to paliperidone. In a total sample of 23 cases with abnormal CYP2D6 enzyme activity 10 cases were identified as poor metabolizers and 12 were identified as intermediate metabolizers.

Discussion

This case series provides clinical examples illustrating the utility of a personalized medicine approach to treatment of irritability in individual with ASD and other neurodevelopmental disorders. In this series, Applicant demonstrated three youth with ASD that demonstrated improvement in severe behaviors from paliperidone treatment as guided by pharmacogenetics testing indicating the individuals were poor metabolizers of aripiprazole and risperidone. The transition to paliperidone was well tolerated and without moderate or severe ADRs. All patients demonstrated a significant clinical improvement based on clinician impression, with a CGI-I score with paliperidone treatment of 2 (“much improved”) or 1 (“very much improved”).

Risperidone is predominately metabolized by CYP2D6 into an active metabolite, 9-hydroxyrisperidone (9-OHR) (Jose de Leon, Wynn, & Sandson, 2010). Unlike risperidone, 9-OHR is primarily excreted unaltered in urine with minimal involvement of hepatic metabolism through 2D6 or 3A4 (Jose de Leon et al., 2010). In the US, 9-OHR is marketed under the name paliperidone for schizophrenia or schizoaffective disorder and, in an oral form, exclusively available in a delayed release formulation (extended release [ER], 1.5-, 3-, 6-, 9-mg tablets). Similar to the parent compound, several studies have demonstrated the efficacy of paliperidone extended release for irritability in individuals with autism (Kowalski et al., 2011; Stigler, Erickson, Mullett, Posey, & McDougle, 2010; Stigler, Mullett, Erickson, Posey, & McDougle, 2012).

Youngster and colleagues recently described two children with ASD (mean age 11.5 years) and a poor CYP2D6 metabolizer phenotype out of a larger cohort of forty subjects who demonstrated significant ADR (severe weight gain and akathisia) and hyperprolactinaemia while being treated with risperidone (Youngster et al., 2014). Blood levels of risperidone in these two PM were nearly nine-fold higher than children with a non-poor CYP2D6 metabolizer phenotype. In addition, the ratio of risperidone/9-OHR was 4.5 compared to non-poor CYP2D6 metabolizers ranged from 0.04 to 0.05. (Youngster et al., 2014).

The use of preemptive CYP2D6 pharmacogenetics testing to specifically target paliperidone in high risk youth with autism may represent a potential cost-effective treatment avenue. In patients who may have severe symptoms of self-injury, aggression and irritability, aspects such as dosing feasibility, length of time to symptom stabilization, tolerability due to ADRs, and treatment efficacy are critical factors in reducing morbidity and avoiding hospitalization (McGuire et al., 2015). The results of the randomized controlled trials (RCTs) that led to FDA approval of risperidone and aripiprazole for irritability in children with autism significantly separated from placebo, but only had an overall improvement rate of 50 to 67% (Robb, 2010). In a recent meta-analysis of thirty-five RCTs with over 1600 participants to assess effective pharmacotherapy for irritability in ASD (Fung et al., 2016) both risperidone and aripiprazole demonstrated a high level of evidence for efficacy in treating irritability in youth with ASD with a number needed to treat (NNT) of 2 and 3, respectively. The number needed to harm (NNH) for adverse effects including sedation, extrapyramidal symptoms, and weight gain for risperidone and aripiprazole ranged between 2 and 20. Interestingly, the NNH was worse than comparable RCTs in children with bipolar disorder, which the authors speculate may reflect specific challenges in the ASD population.

Overall the results of these trials suggest that there remains a significant population of individuals with ASD that will fail first-line treatment with antipsychotics either due to efficacy or tolerability with presumably a fraction of these individual with abnormal 2D6/3A4 metabolism. The feasibility of rapid, simplified methods of isolated CYP2D6/3A4 genotyping is increasingly becoming cost-effective (Langaee, Hamadeh, Chapman, Gums, & Johnson, 2015) and similar strategies are being employed for the treatment of psychiatric conditions in other specialized populations (Berm et al., 2015). In the case an individual with ASD with severe behaviors who is an abnormal CYP2D6 or CYP3A4 metabolizer, it may be that first line treatment with paliperidone may be an optimal treatment approach.

Though there has not been an RCT of paliperidone in ASD for irritability, adult RCTs in schizophrenia suggest paliperidone may offer similar efficacy and potentially slightly decreased rate of adverse effects compared to risperidone (Cai, Lu, Bai, Wu, & Zhao, 2015; Canuso et al., 2008; Nussbaum & Stroup, 2012; Turkoz, Bossie, Lindenmayer, Schooler, & Canuso, 2011). Comparable findings for paliperidone compared to risperidone and aripiprazole have been found in several open label and RCT pediatric trials (Fernandez-Mayoralas et al., 2012; Savitz, Lane, Nuamah, Gopal, & Hough, 2015), if these findings can be replicated in RCTs of individuals with ASD remains to be seen.

Paliperidone may offer additional advantages to risperidone including an extended release daily formulation to improve compliance and the availability of an intramuscular depot shot (paliperidone palmitate) in 1- and 3-month formulations (Alphs et al., 2015; Berwaerts et al., 2015).

Further, though the majority of healthy children are able to learn how to swallow pills, children with ASD with deficits in cognition and communication will have increased difficulty. Thus, the instant invention addresses an important gap in children and individual who cannot swallow capsules by using a reformulation of paliperidone into an extended release sprinkle or an oral suspension formulation.

The availability of an efficacious risperidone metabolite, paliperidone, that largely bypasses hepatic metabolism, suggests a role for a preemptive, cost-effective CYP2D6/3A4 genotype testing to target antipsychotic treatment in individuals with ASD. This approach may provide improvement in the delivery of care to those individual with barriers such as intellectual disability and communication impairments which may be otherwise difficult to monitor for treatment response and adverse effects.

TABLE 1
Summary of clinical and pharmacogenetics features of three case reports.

| | | | Dose | Dose | | | | | |
|---|---|---|---|---|---|---|---|---|---|
| Patient | Sex | Age | (mg) | (mg/kg) | CYP2D6 | AS | CGI-S | CGI-I | AEs |
| 1 | M | 15 | 9 | 0.13 | *4/*9 | 0.5 | 5 | 1 | None |
| 2 | M | 14 | 9 | 0.12 | *4/*17 | 0.5 | 4 | 2 | Appetite |
| 3 | F | 9 | 6 | 0.19 | *4/*5 | 0 | 5 | 2 | None |

M, male; F, female; Dose, paliperidone dose; CYP, cytochrome-P450; 2D6 AS, CYP2D6 Activity Score; CGI-S, Clinical Global Impressions Severity Scale; CGI-I, Clinical Global Impressions Improvement Scale; AEs, Adverse effects; Appetite, Appetite increase.

### REFERENCES

Alphs, L., Benson, C., Cheshire-Kinney, K., Lindenmayer, J. P., Mao, L., Rodriguez, S. C., & Starr, H. L. (2015). Real-world outcomes of aliperidone palmitate compared to daily oral antipsychotic therapy in schizophrenia: a randomized, open-label, review board-blinded 15-month study. J Clin Psychiatry, 76(5), 554-561. doi:10.4088/JCP.14m09584

Berm, E. J. J., Hak, E., Postma, M., Boshuisen, M., Breuning, L., Brouwers, J. R. B. J., . . . Wilffert, B. (2015). Effects and cost-effectiveness of pharmacogenetic screening for CYP2D6 among older adults starting therapy with nortriptyline or venlafaxine: study protocol for a pragmatic randomized controlled trial (CYSCEtrial). Trials, 16, 37. doi:10.1186/s13063-015-0561-0

Berwaerts, J., Liu, Y., Gopal, S., Nuamah, I., Xu, H., Savitz, A., . . . Hough, D. W. (2015). Efficacy and Safety of the 3-Month Formulation of Paliperidone Palmitate vs Placebo for Relapse Prevention of Schizophrenia: A Randomized Clinical Trial. JAMA Psychiatry, 72(8), 830-839. doi: 10.1001/jamapsychiatry.2015.0241

Buitelaar, J. K. (2003). Why have drug treatments been so disappointing. Autism: Neural basis and treatment possibilities, 235-249.

Cai, S., Lu, H., Bai, Z., Wu, R., & Zhao, J. (2015). Paliperidone extended-release tablets in Chinese patients with schizophrenia: meta-analysis of randomized controlled trials. Neuropsychiatr Dis Treat, 11, 1817-1834. doi:10.2147/ndt.s84833

Canuso, C. M., Youssef, E. A., Bossie, C. A., Turkoz, I., Schreiner, A., & Simpson, G. M. (2008). Paliperidone extended-release tablets in schizophrenia patients previously treated with risperidone. Int Clin Psychopharmacol, 23(4), 209-215. doi:10.1097/YIC.0b013e3282fce651

Crews, K. R., Gaedigk, A., Dunnenberger, H. M., Leeder, J. S., Klein, T. E., Caudle, K. E., . . . Skaar, T. C. (2014). Clinical Pharmacogenetics Implementation Consortium guidelines for cytochrome P450 2D6 genotype and codeine therapy: 2014 update. Clin Pharmacol Ther, 95(4), 376-382. doi:10.1038/clpt.2013.254

de Leon, J., Susce, M. T., Pan, R. M., Fairchild, M., Koch, W. H., & Wedlund, P. J. (2005). The CYP2D6 poor metabolizer phenotype may be associated with risperidone adverse drug reactions and discontinuation. J Clin Psychiatry, 66(1), 15-27.

de Leon, J., Wynn, G., & Sandson, N. B. (2010). The Pharmacokinetics of Paliperidone Versus Risperidone. Psychosomatics, 51(1), 80-88. doi:http://dx.doi.org/10.1016/S0033-3182(10)70664-2

Dunnenberger, H. M., Crews, K. R., Hoffman, J. M., Caudle, K. E., Broeckel, U., Howard, S. C., . . . Relling, M. V. (2015). Preemptive Clinical Pharmacogenetics Implementation: Current programs in five United States medical centers. Annual review of pharmacology and toxicology, 55, 89.

Fernandez-Mayoralas, D. M., Fernandez-Jaen, A., Munoz-Jareno, N., Calleja-Perez, B., Fernandez-Perrone, A. L., & Arribas, S. L. (2012). Treatment with paliperidone in children with behavior disorders previously treated with risperidone: an open-label trial. Clin Neuropharmacol, 35(5), 227-230. doi:10.1097/WNF.0b013e31826818cd

Fleeman, N., Martin Saborido, C., Payne, K., Boland, A., Dickson, R., Dundar, Y., . . . Walley, T. (2011). The clinical effectiveness and cost-effectiveness of genotyping for CYP2D6 for the management of women with breast cancer treated with tamoxifen: a systematic review. Health Technol Assess, 15(33), 1-102. doi:10.3310/hta15330

Fung, L. K., Mahajan, R., Nozzolillo, A., Bernal, P., Krasner, A., Jo, B., . . . Hardan, A. Y. (2016). Pharmacologic Treatment of Severe Irritability and Problem Behaviors in Autism: A Systematic Review and Meta-analysis. Pediatrics, 137 Suppl 2, S124-135. doi:10.1542/peds. 2015-2851K

Gaedigk, A., Simon, S., Pearce, R., Bradford, L., Kennedy, M., & Leeder, J. (2008). The CYP2D6 activity score: translating genotype information into a qualitative measure of phenotype. Clinical Pharmacology & Therapeutics, 83(2), 234-242.

Guy, W. (1976). ECDEU Assessment Manual for Psychopharmacology: 1976: National Institute of Mental Health.

Kowalski, J. L., Wink, L. K., Blankenship, K., Habenicht, C. D., Erickson, C. A., Stigler, K. A., & McDougle, C. J. (2011). Paliperidone palmitate in a child with autistic disorder. Journal of child and adolescent psychopharmacology, 21(5), 491-493.

Langaee, T., Hamadeh, I., Chapman, A. B., Gums, J. G., & Johnson, J. A. (2015). A novel simple method for determining CYP2D6 gene copy number and identifying allele(s) with duplication/multiplication. PloS one, 10(1), e0113808. doi:10.1371/journal.pone.0113808

McGuire, K., Erickson, C., Gabriels, R. L., Kaplan, D., Mazefsky, C., McGonigle, J., . . . Siegel, M. (2015). Psychiatric Hospitalization of Children With Autism or Intellectual Disability: Consensus Statements on Best Practices. J Am Acad Child Adolesc Psychiatry, 54(12), 969-971. doi:10.1016/j.jaac.2015.08.017

Nussbaum, A. M., & Stroup, T. S. (2012). Paliperidone palmitate for schizophrenia. Cochrane Database Syst Rev, 6, Cd008296. doi:10.1002/14651858.CD008296.pub2

Patsopoulos, N. A., Ntzani, E. E., Zintzaras, E., & Ioannidis, J. P. (2005). CYP2D6 polymorphisms and the risk of tardive dyskinesia in schizophrenia: a meta-analysis. Pharmacogenet Genomics, 15(3), 151-158.

Robb, A. S. (2010). Managing irritability and aggression in autism spectrum disorders in children and adolescents. Developmental disabilities research reviews, 16(3), 258-264. doi:10.1002/ddrr.118

Roberts, R. L., Mulder, R. T., Joyce, P. R., Luty, S. E., & Kennedy, M. A. (2004). No evidence of increased adverse drug reactions in cytochrome P450 CYP2D6 poor metabolizers treated with fluoxetine or nortriptyline. Hum Psychopharmacol, 19(1), 17-23. doi:10.1002/hup.539

Savitz, A. J., Lane, R., Nuamah, I., Gopal, S., & Hough, D. (2015). Efficacy and safety of paliperidone extended release in adolescents with schizophrenia: a randomized, double-blind study. J Am Acad Child Adolesc Psychiatry, 54(2), 126-137.e121. doi:10.1016/j.jaac.2014.11.009

Shi, C., Yan, W., Wang, G., Wang, F., Li, Q., & Lin, N. (2015). Pharmacogenetics-Based versus Conventional Dosing of Warfarin: A Meta-Analysis of Randomized Controlled Trials. PloS one, 10(12), e0144511. doi:10.1371/journal.pone.0144511

Sistonen, J., Sajantila, A., Lao, O., Corander, J., Barbujani, G., & Fuselli, S. (2007). CYP2D6 worldwide genetic variation shows high frequency of altered activity variants and no continental structure. Pharmacogenet Genomics, 17(2), 93-101. doi:10.1097/01.fpc.0000239974.69464.f2

Stigler, K. A., Erickson, C. A., Mullett, J. E., Posey, D. J., & McDougle, C. J. (2010). Paliperidone for irritability in autistic disorder. Journal of child and adolescent psychopharmacology, 20(1), 75-78.

Stigler, K. A., Mullett, J. E., Erickson, C. A., Posey, D. J., & McDougle, C. J. (2012). Paliperidone for irritability in adolescents and young adults with autistic disorder. Psychopharmacology, 223(2), 237-245.

Turkoz, I., Bossie, C. A., Lindenmayer, J. P., Schooler, N., & Canuso, C. M. (2011). Paliperidone ER and oral risperidone in patients with schizophrenia: a comparative database analysis. BMC psychiatry, 11, 21. doi:10.1186/1471-244x-11-21

Vuilleumier, P. H., Stamer, U. M., & Landau, R. (2012). Pharmacogenomic considerations in opioid analgesia. Pharmacogenomics and Personalized Medicine, 5, 73-87. doi:10.2147/PGPM.S23422

Youngster, I., Zachor, D. A., Gabis, L. V., Bar-Chaim, A., Benveniste-Levkovitz, P., Britzi, M., . . . Berkovitch, M. (2014). CYP2D6 genotyping in paediatric patients with autism treated with risperidone: a preliminary cohort study. Developmental Medicine & Child Neurology, 56(10), 990-994.

Zhou, S.-F. (2009). Polymorphism of human cytochrome P450 2D6 and its clinical significance. Clinical pharmacokinetics, 48(12), 761-804.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of treating a neurodevelopmental disorder (NDD) associated irritability in an individual in need thereof, comprising the step of administering to said individual a formulation comprising 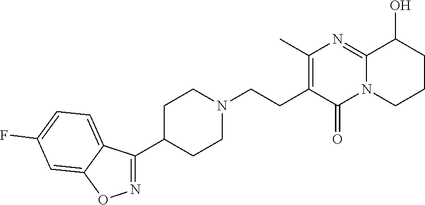 [SMILES: Cc1nc2n(c(=O)c1CCN1CCC(c3noc4cc(F)ccc34)CC1)CCCC2O]
(Paliperidone)
or a pharmaceutically acceptable salt thereof;
wherein said paliperidone or pharmaceutically acceptable salt thereof is in a sprinkle dosage form.

2. The method of claim 1 wherein said paliperidone or pharmaceutically acceptable salt thereof is administered at an interval selected from once a day, twice a day, three times a day, or four times a day.

3. The method of claim 1, wherein said individual is administered a dose of about 1.5 to about 18 mg/day, or about 3 to about 15 mg/day, or about 5 to about 12 mg/day.

4. The method of claim 1 wherein said sprinkle dosage form is in a form selected from an immediate release form and a controlled-release formulation.

5. The method of claim 4 wherein said sprinkle dosage form is taste-masked.

6. A method of diagnosing and treating a symptom of a neurodevelopmental disorder (NDD), in an individual in need thereof comprising the steps of:
a. detecting whether a mutation exists in a biological sample obtained from said individual, wherein said mutation causes reduced activity of one or both of CYP2D6 and CYP3A4 activity in said individual;
b. diagnosing said individual as a candidate for paliperidone treatment when the presence of said mutation is detected; and
c. administering an effective amount of paliperidone or pharmaceutically acceptable salt thereof to said individual diagnosed as a candidate for paliperidone treatment.

7. The method of claim 6, wherein said symptom is selected from one or more of irritability, self-injurious behavior, intermittent explosive disorder, disruptive behavior disorder, and aggression.

8. The method of claim 6, wherein said NDD is selected from a developmental disorder (DD), an intellectual disability (ID), global developmental delay (GDD) or a combination thereof.

9. The method of claim 6, wherein said paliperidone or pharmaceutically acceptable salt thereof is administered in a sprinkle dosage form.

10. The method of claim 9 wherein said sprinkle dosage form comprises a capsule enclosing a plurality of coated granules or microcapsules, wherein said capsule has a means for opening said capsule for release of said plurality of coated granules or microcapsules.

11. The method of claim 10 wherein said sprinkle dosage form is taste-masked.

12. The method of claim 6, wherein said mutation results in a reduced capacity to metabolize one or both of risperidone and aripiprazole.

13. A method of diagnosing and treating a symptom selected from irritability, tantrums marked with excessive duration or severity, intermittent explosive disorder, bipolar affective disorder, disruptive behavior disorder, major depressive disorder, aggression, catatonic disorder, and psychotic disorders, or combinations thereof, in an individual diagnosed with a Neurodevelopmental Disorder (NDD), said method comprising the steps of
a. obtaining a biological sample from a human patient;
b. detecting the activity of CYP 2D6 and CYP 3A4;
c. diagnosing the patient as a candidate for paliperidone treatment when the activity of one or both of CYP 2D6 and CYP3A4 is reduced or expected to be reduced based on the presence of a mutation in a gene selected from one or both of CYP 2D6 and CYP3A4 that is expected to disrupt function of one or both of CYP 2D6 and CYP3A4; and
d. administering an effective amount of paliperidone or pharmaceutically acceptable salt thereof to the diagnosed individual, wherein said paliperidone or pharmaceutically acceptable salt thereof is in a sprinkle dosage form.

14. A kit comprising
a. paliperidone or pharmaceutically acceptable salt thereof and a pharmaceutically acceptable carrier, wherein said paliperidone or pharmaceutically acceptable salt thereof is in a sprinkle dosage form; and
b. a means for delivery of the composition to a human.

15. An article of manufacture comprising:
a. a container comprising a label; and
b. a composition comprising paliperidone or pharmaceutically acceptable salt thereof and a pharmaceutically acceptable excipient, wherein the label indicates that the composition is to be administered to an individual diagnosed with an autism spectrum disorder (ASD) and having a symptom selected from irritability, self-injurious behavior, intermittent explosive disorder, disruptive behavior disorder, and aggression.

16. A composition comprising paliperidone having the following structure 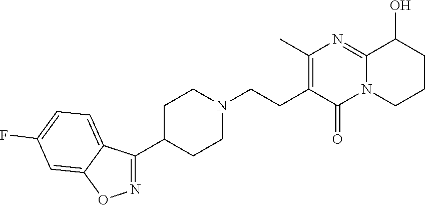 [SMILES: Cc1nc2n(c(=O)c1CCN1CCC(c3noc4cc(F)ccc34)CC1)CCCC2O]
(Paliperidone)
or a pharmaceutically acceptable salt thereof, for treatment of a neurodevelopmental disorder (NDD, wherein said medicament is in a sprinkle dosage form.

17. The composition of claim 16 wherein said paliperidone or pharmaceutically acceptable salt thereof is administered at an interval selected from once a day, twice a day, three times a day, or four times a day, wherein said paliperidone is in a dose of from about 1.5 to about 18 mg/day, or about 3 to about 15 mg/day, or about 5 to about 12 mg/day, wherein said medicament is a sprinkle dosage form selected from an immediate release form and a controlled-release formulation.

18. The composition of claim 16, wherein said composition is administered to an individual having a neurodevelopmental disorder (NDD), wherein said individual has a mutation causing reduced activity in one or both of CYP2D6 and CYP3A4, wherein said paliperidone is administered in an amount sufficient to treat one or more conditions selected from irritability, self-injurious behavior, intermittent explosive disorder, disruptive behavior disorder, and aggression.

19. The method of claim 16, wherein said coated granules or microcapsules are enclosed in a capsule, wherein said capsule has a means for opening said capsule for release of said coated granules or microcapsules.

20. A method of diagnosing and treating a symptom selected from irritability, tantrums marked with excessive duration or severity, intermittent explosive disorder, bipolar affective disorder, disruptive behavior disorder, major depressive disorder, aggression, catatonic disorder, and psychotic disorders, or combinations thereof, in an individual diagnosed with a Neurodevelopmental Disorder (NDD), said method comprising the steps of
a. obtaining a biological sample from a human patient;
b. detecting the activity of CYP 2D6 and CYP 3A4;
c. diagnosing the patient as a candidate for paliperidone treatment when the activity of one or both of CYP 2D6 and CYP3A4 is reduced or expected to be reduced based on the presence of a mutation in one or both of CYP 2D6 and CYP3A4 that is expected to disrupt function of one or both of CYP 2D6 and CYP3A4; and
d. administering an effective amount of paliperidone or pharmaceutically acceptable salt thereof to the diagnosed individual, wherein said paliperidone is in a sprinkle dosage form.
